(19) United States
(12) Patent Application Publication
(10) Pub. No.: US 2025/0388528 A1
(43) Pub. Date: Dec. 25, 2025

(54) QUATERNARY AMMONIUM SALTS AND METHODS OF MAKING THEREOF

(71) Applicant: Bio Protectant Technologies, Inc., Dover, DE (US)

(72) Inventor: Chad Joseph Roy, San Diego, CA (US)

(21) Appl. No.: 19/060,579

(22) Filed: Feb. 21, 2025

Related U.S. Application Data
(60) Provisional application No. 63/556,825, filed on Feb. 22, 2024.

(51) Int. Cl. C07C 209/12; C07C 211/63

(52) U.S. Cl. CPC C07C 209/12; C07C 211/63

(57) ABSTRACT

Provided herein, in some embodiments, are quaternary ammonium salt compositions with one or more improved characteristics over other quaternary ammonium salt compositions. In some embodiments, those characteristics are one or combination of less impurity and better homogeneity of alkyl length. Also provided herein, in some embodiments, are methods of making the quaternary ammonium salt compounds with one or more improved characteristics over other methods of making the quaternary ammonium salt compounds including, but not limited to, better processability, better yield, better purity, or combinations thereof.

### RELATED APPLICATIONS

This application claims the benefit of U.S. Provisional Application No. 63/556,825, filed on Feb. 22, 2024, of which is incorporated herein by reference in its entirety.

### SUMMARY OF THE DISCLOSURE

One aspect of the present disclosure relates to a method for producing a compound of Formula 1:

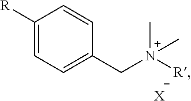

wherein the R is hydrogen or a C1-C4 alkyl, wherein the R′ is a C4-C22 alkyl, and wherein the X is a halogen, the method comprising: dissolving an alkyl dimethyl amine R′N(CH₃)₂ and a halide according to Formula 2:

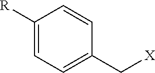

in a solvent to form a reaction mixture, wherein the solvent is ethanol, ethyl acetate, or a mixture thereof; heating the reaction mixture at a reaction temperature less than the reflux temperature of the solvent; and allowing the reaction mixture to cool, thereby obtaining a precipitate of alkyl dimethyl 4-ethylbenzyl ammonium from the reaction mixture.

In some embodiments, the R is selected from the group consisting of hydrogen, methyl, or ethyl. In some embodiments, the R is selected from the group consisting of hydrogen or ethyl. In some embodiments, the R is ethyl.

In some embodiments, the R′ is a C4-C20 alkyl. In some embodiments, the R′ is a C6-C20 alkyl. In some embodiments, the R′ is a C8-C20 alkyl. In some embodiments, the R′ is a C10-C20 alkyl. In some embodiments, the R′ is a C12-C20 alkyl. In some embodiments, the R′ is a C4-C18 alkyl. In some embodiments, the R′ is a C6-C18 alkyl. In some embodiments, the R′ is a C8-C18 alkyl. In some embodiments, the R′ is a C10-C18 alkyl. In some embodiments, the R′ is a C12-C18 alkyl. In some embodiments, the R′ is a C4-C16 alkyl. In some embodiments, the R′ is a C6-C16 alkyl. In some embodiments, the R′ is a C8-C16 alkyl. In some embodiments, the R′ is a C10-C16 alkyl. In some embodiments, the R′ is a C12-C16 alkyl. In some embodiments, the R′ is a C12 or C14 alkyl. In some embodiments, the R′ is a C12 alkyl. In some embodiments, the R′ is dodecyl.

In some embodiments, the halide is chloride or bromide. In some embodiments the halide is chloride.

In some embodiments, the reaction mixture is heated at a temperature of from about 30° C. to about 60° C. In some embodiments, the reaction mixture is heated at a temperature of from about 30° C. to about 70° C. In some embodiments, the reaction mixture is heated at a temperature of from about 30° C. to about 65° C. In some embodiments, the reaction mixture is heated at a temperature of from about 30° C. to about 55° C. In some embodiments, the reaction mixture is heated at a temperature of from about 35° C. to about 75° C. In some embodiments, the reaction mixture is heated at a temperature of from about 35° C. to about 70° C. In some embodiments, the reaction mixture is heated at a temperature of from about 35° C. to about 65° C. In some embodiments, the reaction mixture is heated at a temperature of from about 35° C. to about 60° C. In some embodiments, the reaction mixture is heated at a temperature of from about 35° C. to about 55° C. In some embodiments, the reaction mixture is heated at a temperature of from about 40° C. to about 75° C. In some embodiments, the reaction mixture is heated at a temperature of from about 40° C. to about 70° C. In some embodiments, the reaction mixture is heated at a temperature of from about 45° C. to about 75° C. In some embodiments, the reaction mixture is heated at a temperature of from about 45° C. to about 70° C. In some embodiments, the reaction mixture is heated at a temperature of from about 45° C. to about 60° C. In some embodiments, the reaction mixture is heated at a temperature of from about 45° C. to about 55° C. In some embodiments, the reaction mixture is heated at a temperature of from about 50° C. to about 75° C. In some embodiments, the reaction mixture is heated at a temperature of from about 50° C. to about 70° C. In some embodiments, the reaction mixture is heated at a temperature of from about 50° C. to about 65° C. In some embodiments, the reaction mixture is heated at a temperature of from about 50° C. to about 60° C. In some embodiments, the reaction mixture is heated at a temperature of from about 50° C. to about 55° C. In some embodiments, the reaction mixture is heated at a temperature of from about 55° C. to about 75° C. In some embodiments, the reaction mixture is heated at a temperature of from about 55° C. to about 65° C. In some embodiments, the reaction mixture is heated at a temperature of from about 55° C. to about 60° C.

In some embodiments, wherein the solvent is anhydrous ethanol. In some embodiments, wherein the solvent is ethyl acetate.

In some embodiments, wherein the solvent is anhydrous ethanol, the reaction mixture is heated at a temperature of from about 55° C. to about 65° C. In some embodiments, wherein the solvent is ethyl acetate, the reaction mixture is heated at a temperature of from about 40° C. to about 55° C.

In some embodiments, the heating of the reaction mixture continues until less than 20% R′N(CH₃)₂ is present in the reaction mixture. In some embodiments, the heating of the reaction mixture continues until less than 15% R′N(CH₃)₂ is present in the reaction mixture. In some embodiments, the heating of the reaction mixture continues until less than 10% R′N(CH₃)₂ is present in the reaction mixture. In some embodiments, the heating of the reaction mixture continues until less than 5% R′N(CH₃)₂ is present in the reaction mixture. In some embodiments, the heating of the reaction mixture continues until less than 1% R′N(CH₃)₂ is present in the reaction mixture. In some embodiments, the heating of the reaction mixture continues until less than 0.1% R′N(CH₃)₂ is present in the reaction mixture.

In some embodiments, the reaction is carried out for at least 1 hour to about 48 hours. In some embodiments, the reaction is carried out for at least 1 hour to about 36 hours. In some embodiments, the reaction is carried out for at least 1 hour to about 30 hours. In some embodiments, the reaction is carried out for at least 1 hour to about 24 hours. In some embodiments, the reaction is carried out for at least 1 hour to about 18 hours. In some embodiments, the reaction is carried out for at least 1 hour to about 12 hours. In some embodiments, the reaction is carried out for at least 1 hour to about 6 hours.

In some embodiments, the precipitate is washed with ethyl acetate. In some embodiments, the precipitate is washed until achieving a purity of greater than 99%.

In some embodiments, the precipitate is dried under vacuum at a temperature of from about 20° C. to about 45° C. In some embodiments, the precipitate is dried under vacuum at a temperature of from about 20° C. to about 40° C. In some embodiments, the precipitate is dried under vacuum at a temperature of from about 20° C. to about 35° C. In some embodiments, the precipitate is dried under vacuum at a temperature of from about 20° C. to about 30° C. In some embodiments, the precipitate is dried under vacuum at a temperature of from about 25° C. to about 45° C. In some embodiments, the precipitate is dried under vacuum at a temperature of from about 25° C. to about 40° C. In some embodiments, the precipitate is dried under vacuum at a temperature of from about 25° C. to about 35° C. In some embodiments, the precipitate is dried under vacuum at a temperature of from about 25° C. to about 30° C. In some embodiments, the precipitate is dried under vacuum at a temperature of from about 30° C. to about 45° C. In some embodiments, the precipitate is dried under vacuum at a temperature of from about 30° C. to about 40° C. In some embodiments, the precipitate is dried under vacuum at a temperature of from about 30° C. to about 35° C. In some embodiments, the precipitate is dried under vacuum at a temperature of from about 30° C. to about 33° C.

In some embodiments, the reaction mixture is essentially free of water. In some embodiments, the reaction mixture contains less than 15% of water. In some embodiments, the reaction mixture contains less than 10% of water. In some embodiments, the reaction mixture contains less than 5% of water. In some embodiments, the reaction mixture contains less than 3% of water. In some embodiments, the reaction mixture contains less than 2% of water. In some embodiments, the reaction mixture contains less than 1% of water. In some embodiments, the reaction mixture contains less than 0.5% of water. In some embodiments, the reaction mixture contains less than 0.1% of water.

In some embodiments, the molar ratio of R′N(CH₃)₂ and the halide according to Formula 2 is about 1:1.

In some embodiments, the concentration of ethyl acetate is about 190 g/ml to about 200 g/ml.

Another aspect of the present disclosure relates to a compound according to Formula 1 made by any of the methods disclosed herein.

Another aspect of the present disclosure relates to a composition, wherein the composition comprises a quaternary ammonium salt.

Disclosed herein, in some embodiments, is a composition, wherein at least 85% of the composition is the compound according to Formula 1. Disclosed herein, in some embodiments, is a composition, wherein at least 90% of the composition is the compound according to Formula 1. Disclosed herein, in some embodiments, is a composition, wherein at least 95% of the composition is the compound according to Formula 1. Disclosed herein, in some embodiments, is a composition, wherein at least 98% of the composition is the compound according to Formula 1. Disclosed herein, in some embodiments, is a composition, wherein at least 99% of the composition is the compound according to Formula 1.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE FIGURES

FIG. 1 illustrates ¹HNMR spectrum of alkyl dimethyl benzyl ammonium chloride.

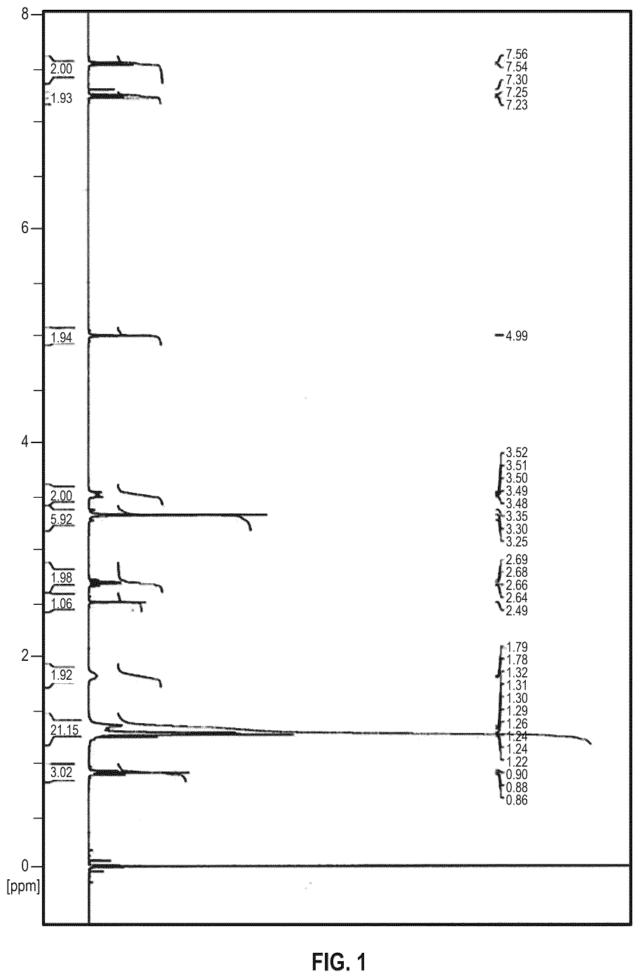

FIG. 2 illustrates UV chromatogram for a solvent blank at 220 nm.

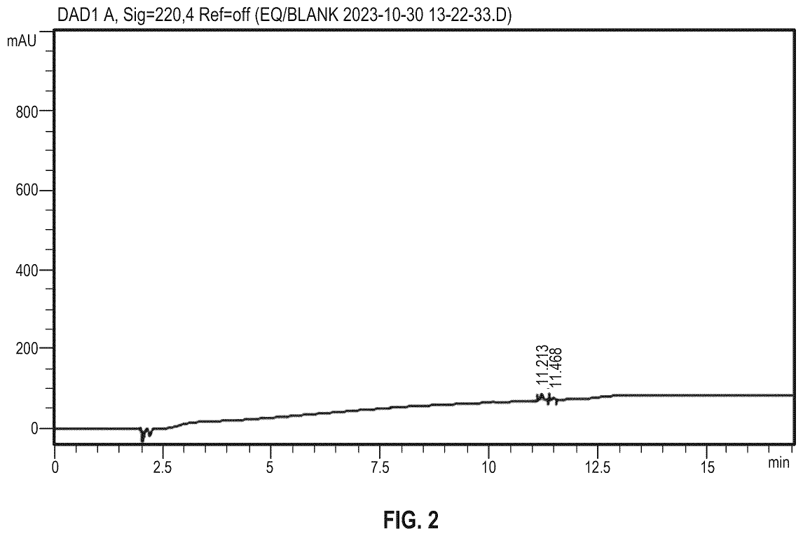

FIG. 3 illustrates UV chromatogram of alkyl dimethyl benzyl ammonium chloride at 220 nm.

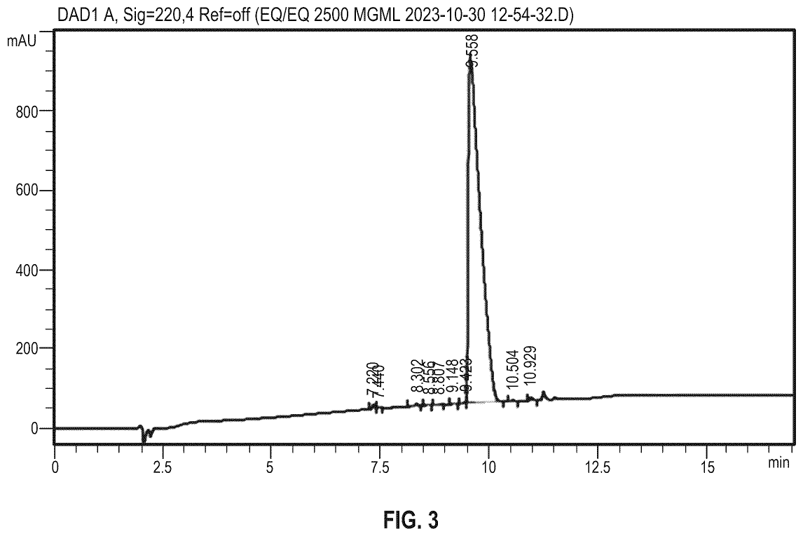

FIG. 4 illustrates Area Percentage for UV at 220 nm.

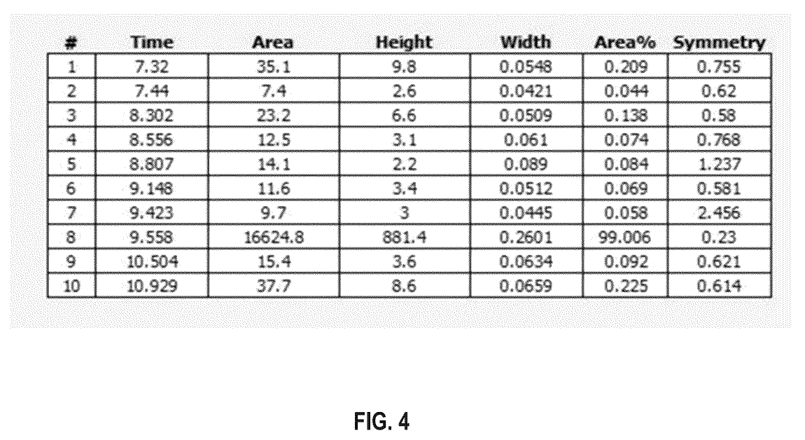

FIG. 5 illustrates Mass Spectrum of alkyl dimethyl benzyl ammonium chloride. [M]⁺ at 332.33 m/z is noted.

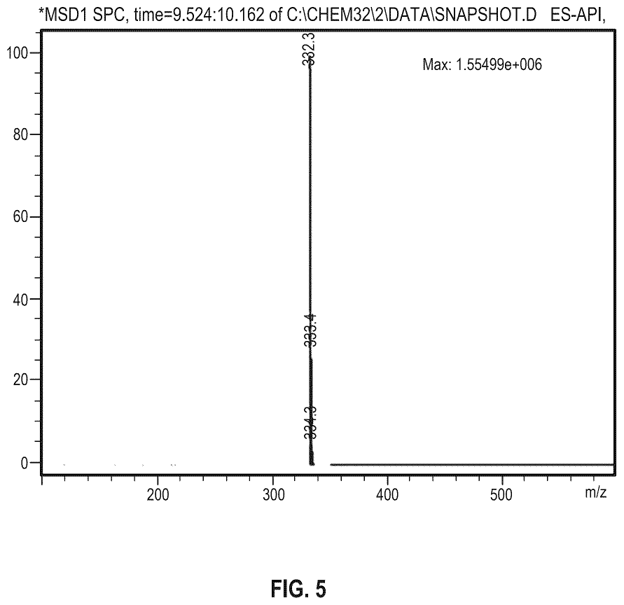

### DETAILED DESCRIPTION OF THE DISCLOSURE

Provided herein, in some embodiments, are quaternary ammonium salt compositions with one or more improved characteristics over other quaternary ammonium salt compositions. In some embodiments, those characteristics are one or combination of less impurity, homogeneity of alkyl length, and others. Also provided herein, in some embodiments, are methods of making the quaternary ammonium salt compounds with one or more improved characteristics over other methods of making the quaternary ammonium salt compounds including, but not limited to, better processability, better yield, better purity, or combinations thereof.

Detailed descriptions of one or more embodiments are provided herein. It is to be understood, however, that the present disclosure is embodied in various forms. Therefore, specific details disclosed herein are not to be interpreted as limiting, but rather as a basis for the claims and as a representative basis for teaching one skilled in the art to employ the present disclosure in any appropriate manner.

Wherever the phrase “for example,” “such as,” “including,” and the like are used herein, the phrase “and without limitation” is understood to follow unless explicitly stated otherwise. Similarly, “an example,” “exemplary,” and the like are understood to be non-limiting.

The term “substantially” allows for deviations from the descriptor that does not negatively impact the intended purpose. Descriptive terms are understood to be modified by the term “substantially” even if the word “substantially” is not explicitly recited. Therefore, for example, the phrase “wherein the level extends vertically” means “wherein the lever extends substantially vertically” so long as a precise vertical arrangement is not necessary for the level to perform its function.

The term “about” is used to indicate that a value includes the standard level of error for the device or method being employed to determine the value. In some embodiments, the level of error is 10%. In some embodiments, the level of error is 9%. In some embodiments, the level of error is 8%. In some embodiments, the level of error is 7%. In some embodiments, the level of error is 6%. In some embodiments, the level of error is 5%.

The terms “comprising,” “including,” “having,” “involving,” (and similarly “comprises,” “includes,” “has,” and “involves”), and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b, and c. Whenever the terms “a” or “an” are used, “one or more” is understood, unless such interpretation is nonsensical in context.

### Quaternary Compounds

Quaternary compounds, such as benzalkonium salts, constitute an economically important class of industrial chemicals that are widely distributed among a diverse array of products and users from an industrial to the household sector. The present disclosure recognizes that quaternary compounds can be used as disinfectants, biocides, and detergents. Quaternary compounds can also be used as anti-electrostatics and as phase transfer catalysts.

### Quaternary Ammonium Salts

Quaternary ammonium compounds (QACs) are a group of chemicals commonly found in disinfectants, preservatives, and surfactants. QACs are widely used in various industrial, commercial, and household applications and are present in hospital-grade disinfectant sprays and wipes, surgical instrument sterilization compounds, laundry detergents, and standing-water treatments. For more than 50 years, QACs have been studied, used, and considered safe for human use.

QACs contain a positively charged ammonium group connected to at least one hydrophobic hydrocarbon. The most commonly used QACs are alkyldimethylbenzyl ammonium chloride (ADBAC or BAC), cetylpyridinium chloride (CPC), dodecyl-dimethyl ammonium chloride (DDAC), and cocobenzyldimethyl ammonium chloride (BKC).

Some aspects of the present disclosure relate to chemical synthesis of quaternary ammonium salts and products thereof. In some aspects, the quaternary ammonium salt is a salt of a quaternary ammonium cation. As used herein “quaternary ammonium cations” refer to positively charged polyatomic ions of the structure NR₄⁺, R being an optionally substituted alkyl group or an optionally substituted aryl group. Unlike the ammonium ion (NH₄⁺) and the primary, secondary, or tertiary ammonium cations, the quaternary ammonium cations are permanently charged, independent of the pH of their solution. In some embodiments, the quaternary ammonium salt is not a polymeric quaternary ammonium salt. In some embodiments, the quaternary ammonium salt comprises a C4-C16 alkyl chain. In some embodiments, the quaternary ammonium salt comprises a C12 or C14 alkyl chain.

In some embodiments, the composition is essentially free of alkyl(ethylbenzyl)dimethyl ammonium salt having an alkyl of less than 12 carbons or more than 14 carbons.

### Benzalkonium Salts

Benzalkonium salts have bactericidal and antimicrobial properties. They are used as preservatives in a variety of products, such as ophthalmic, nasal, and parenteral products. They are also used as topical antiseptics and disinfectants for medical equipment.

Benzalkonium salts, traditionally, are not used as single compounds, but rather as mixtures composed of two or three benzalkonium salts differing only in the length of the alkyl chains. Such mixtures are produced on a large-scale in industry. The present disclosure recognizes that the antimicrobial activity of benzalkonium salts depends on a varying length of the side n-alkyl chain, and recognizes the desirability of a targeted synthesis of pure individual salts such that specificity of each salt against different pathogens can be utilized.

### C4-C16 Alkyl(Ethylbenzyl)Dimethyl Ammonium Salts

The present disclosure recognizes that homologs of benzalkonium salts, such as C4-C16 alkyl(ethylbenzyl)dimethyl ammonium salts, have bactericidal and antimicrobial properties. They are used as preservatives in a variety of products, such as ophthalmic, nasal, and parenteral products. They are also used as topical antiseptics and disinfectants for medical equipment.

Like benzalkonium salts, alkyl(ethylbenzyl)dimethyl ammonium salts are not used as single compounds, but rather as mixtures composed of two or three alkyl(ethylbenzyl)dimethyl ammonium salts differing only in the length of the alkyl chains. Such mixtures are produced on a large-scale in industry. The present disclosure recognizes that the antimicrobial activity of benzalkonium salts depends on a varying length of the side n-alkyl chain, and recognizes the desirability of a targeted synthesis of pure individual salts such that specificity of each salt against different pathogens can be utilized.

### Chemical Synthesis

### Alkyl Dimethyl Amine

An aspect of the present disclosure relates to C4-C20 alkyl dimethyl amines. C4-C20 alkyl dimethyl amines, such as N,N-Dimethyldodecylamine (DMCA) can be reacted to form quaternary ammonium salt compounds. DMCA has the following formula:

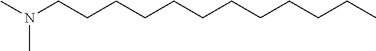

### Benzyl Halide

Another aspect of the present disclosure relates to benzyl halides. The DMCA can be reacted with a benzyl halide of Formula 2:

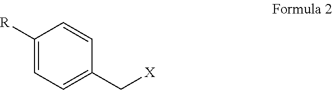

In some embodiments, R is ethyl, methyl, or hydrogen. In some embodiments, X is a halogen.

The synthesis method of the quaternary ammonium salts disclosed herein is provided below using DMCA as a non-limiting example:

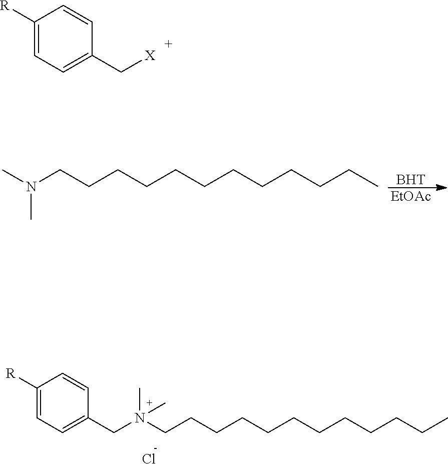

Starting from the reaction between DMCA and benzyl halide, in a solvent, the quaternary ammonium salt is obtained.

According to one aspect of the present disclosure, a DMCA, when refluxed with benzyl halide, form a quaternary ammonium salt with one or more improved characteristics including, but not limited to, better processability, better purity, better yield, or combinations thereof.

### Solvent

### Ethanol

Another aspect of the present disclosure relates to using ethanol, such as anhydrous ethanol, as the solvent for the chemical synthesis disclosed herein. Anhydrous ethanol is a colorless liquid with a molecular mass of 46.1 grams per mole and a boiling point of 78° C.

### Ethyl Acetate

Another aspect of the present disclosure relates to using ethyl acetate as the solvent for the chemical synthesis disclosed herein. Ethyl acetate is a colorless liquid with a molecular weight of 88.406 grams per mole. Ethyl acetate is prone to hydrolysis, transesterification, and condensations and has a boiling point of 77° C.

### Solvent Mixture

Another aspect of the present disclosure relates to using solvent mixtures, such mixture of ethanol and ethyl acetate, as the solvent for the chemical synthesis disclosed herein.

### Initiator

Another aspect of the present disclosure relates to using 2,6-ditertbutyl-4-methylphenol as an initiator of the chemical synthesis disclosed herein. 2,6-ditertbutyl-4-methylphenol is a white crystalline solid. 2,6-ditertbutyl-4-methylphenol has a molecular weight of 220.35 grams per mole, a boiling point of 264° C., and a melting point of 69°-72° C.

### NON-LIMITING EMBODIMENTS

The following embodiments are given to enable those skilled in the art to more clearly understand and to practice the present disclosure. They should not be considered as limiting the scope of the disclosure, but merely as being illustrative and representative thereof.
1. A method making a compound according to Formula 1

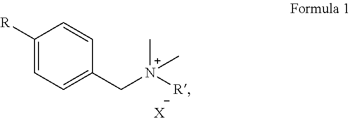

wherein R is hydrogen or a C1-C4 alkyl, wherein R′ is a C4-C22 alkyl, wherein X is a halogen, the method comprising:
(a) dissolving an alkyldimethyl amine R′N(CH₃)₂ and a halide according to Formula 2

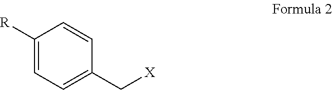

in a solvent to form a reaction mixture, wherein the solvent is anhydrous ethanol, ethyl acetate, or a mixture thereof;
(b) heating the reaction mixture at a reaction temperature less than the reflux temperature of the solvent; and
(c) allowing the reaction mixture to cool, thereby obtaining a precipitate of alkyl dimethyl 4-ethylbenzyl ammonium from the reaction mixture.
2. The method of embodiment 1, wherein R is selected from the group consisting of hydrogen, methyl or ethyl.
3. The method of embodiment 1, wherein R is selected from the group consisting of hydrogen or ethyl.
4. The method of embodiment 1, wherein R is ethyl.
5. The method of embodiment 1, wherein R′ is a C4-C20 alkyl.
6. The method of embodiment 1, wherein R′ is a C6-C20 alkyl.
7. The method of embodiment 1, wherein R′ is a C8-C20 alkyl.
8. The method of embodiment 1, wherein R′ is a C10-C20 alkyl.
9. The method of embodiment 1, wherein R′ is a C12-C20 alkyl. 9.
10. The method of embodiment 1, wherein R′ is a C4-C18 alkyl. 10.
11. The method of embodiment 1, wherein R′ is a C6-C18 alkyl. 11.
12. The method of embodiment 1, wherein R′ is a C8-C16 alkyl. 12.
13. The method of embodiment 1, wherein R′ is a C10-C18 alkyl.
14. The method of embodiment 1, wherein R′ is a C12-C18 alkyl.
15. The method of embodiment 1, wherein R′ is a C4-C16 alkyl.
16. The method of embodiment 1, wherein R′ is a C6-C16 alkyl. 16.
17. The method of embodiment 1, wherein R′ is a C8-C16 alkyl.
18. The method of embodiment 1, wherein R′ is a C10-C16 alkyl.
19. The method of embodiment 1, wherein R′ is a C12-C16 alkyl.
20. The method of embodiment 1, wherein R′ is a C12 or C14 alkyl.
21. The method of embodiment 1, wherein R′ is a C12 alkyl.
22. The method of embodiment 1, wherein R′ is dodecyl.
23. The method of embodiments 1-22, wherein the halide is chloride or bromide.
24. The method of embodiments 1-22, wherein the halide is chloride.
25. The method of embodiments 1-24, wherein the reaction mixture in step (b) is heated at a temperature of from about 30° C. to about 75° C.
26. The method of embodiments 1-24, wherein the reaction mixture in step (b) is heated at a temperature of from about 30° C. to about 70° C.
27. The method of embodiments 1-24, wherein the reaction mixture in step (b) is heated at a temperature of from about 30° C. to about 65° C.
28. The method of embodiments 1-24, wherein the reaction mixture in step (b) is heated at a temperature of from about 30° C. to about 60° C.
29. The method of embodiments 1-24, wherein the reaction mixture in step (b) is heated at a temperature of from about 30° C. to about 55° C.
30. The method of embodiments 1-24, wherein the reaction mixture in step (b) is heated at a temperature of from about 35° C. to about 75° C.
31. The method of embodiments 1-24, wherein the reaction mixture in step (b) is heated at a temperature of from about 35° C. to about 70° C.
32. The method of embodiments 1-24, wherein the reaction mixture in step (b) is heated at a temperature of from about 35° C. to about 65° C.
33. The method of embodiments 1-24, wherein the reaction mixture in step (b) is heated at a temperature of from about 35° C. to about 60° C.
34. The method of embodiments 1-24, wherein the reaction mixture in step (b) is heated at a temperature of from about 35° C. to about 55° C.
35. The method of embodiments 1-24, wherein the reaction mixture in step (b) is heated at a temperature of from about 40° C. to about 75° C.
36. The method of embodiments 1-24, wherein the reaction mixture in step (b) is heated at a temperature of from about 40° C. to about 70° C.
37. The method of embodiments 1-24, wherein the reaction mixture in step (b) is heated at a temperature of from about 40° C. to about 65° C.
38. The method of embodiments 1-24, wherein the reaction mixture in step (b) is heated at a temperature of from about 40° C. to about 60° C.
39. The method of embodiments 1-24, wherein the reaction mixture in step (b) is heated at a temperature of from about 40° C. to about 55° C.
40. The method of embodiments 1-24, wherein the reaction mixture in step (b) is heated at a temperature of from about 45° C. to about 75° C.
41. The method of embodiments 1-24, wherein the reaction mixture in step (b) is heated at a temperature of from about 45° C. to about 70° C.
42. The method of embodiments 1-24, wherein the reaction mixture in step (b) is heated at a temperature of from about 45° C. to about 65° C.
43. The method of embodiments 1-24, wherein the reaction mixture in step (b) is heated at a temperature of from about 45° C. to about 60° C.
44. The method of embodiments 1-24, wherein the reaction mixture in step (b) is heated at a temperature of from about 45° C. to about 55° C.
45. The method of embodiments 1-24, wherein the reaction mixture in step (b) is heated at a temperature of from about 50° C. to about 75° C.
46. The method of embodiments 1-24, wherein the reaction mixture in step (b) is heated at a temperature of from about 50° C. to about 70° C.
47. The method of embodiments 1-24, wherein the reaction mixture in step (b) is heated at a temperature of from about 50° C. to about 65° C.
48. The method of embodiments 1-24, wherein the reaction mixture in step (b) is heated at a temperature of from about 50° C. to about 60° C.
49. The method of embodiments 1-24, wherein the reaction mixture in step (b) is heated at a temperature of from about 50° C. to about 55° C.
50. The method of embodiments 1-24, wherein the reaction mixture in step (b) is heated at a temperature of from about 55° C. to about 75° C.
51. The method of embodiments 1-24, wherein the reaction mixture in step (b) is heated at a temperature of from about 55° C. to about 70° C.
52. The method of embodiments 1-24, wherein the reaction mixture in step (b) is heated at a temperature of from about 55° C. to about 65° C.
53. The method of embodiments 1-24, wherein the reaction mixture in step (b) is heated at a temperature of from about 55° C. to about 60° C.
54. The method of embodiments 1-24, wherein the solvent is anhydrous ethanol.
55. The method of embodiments 1-24, wherein the solvent is ethyl acetate.
56. The method of embodiment 54, wherein the reaction mixture in step (b) is heated at a temperature of from about 55° C. to about 65° C.
57. The method of embodiment 55, wherein the reaction mixture in step (b) is heated at a temperature of from about 40° C. to about 55° C.
58. The method of embodiments 1-57, wherein the heating continues until less than 20% R′N(CH₃)₂ is present in the reaction mixture.
59. The method of embodiments 1-57, wherein the heating continues until less than 15% R′N(CH₃)₂ is present in the reaction mixture.
60. The method of embodiments 1-57, wherein the heating continues until less than 10% R′N(CH₃)₂ is present in the reaction mixture.
61. The method of embodiments 1-57, wherein the heating continues until less than 5% R′N(CH₃)₂ is present in the reaction mixture.
62. The method of embodiments 1-57, wherein the heating continues until less than 1% R′N(CH₃)₂ is present in the reaction mixture.
63. The method of embodiments 1-57, wherein the heating continues until less than 0.1% R′N(CH₃)₂ is present in the reaction mixture.
64. The method of embodiments 1-63, wherein the reaction is carried out for at least 1 hour to about 48 hours.
65. The method of embodiments 1-63, wherein the reaction is carried out for at least 1 hour to about 36 hours.
66. The method of embodiments 1-63, wherein the reaction is carried out for at least 1 hour to about 30 hours.
67. The method of embodiments 1-63, wherein the reaction is carried out for at least 1 hour to about 24 hours.
68. The method of embodiments 1-63, wherein the reaction is carried out for at least 1 hour to about 18 hours.
69. The method of embodiments 1-63, wherein the reaction is carried out for at least 1 hour to about 12 hours.
70. The method of embodiments 1-63, wherein the reaction is carried out for at least 1 hour to about 6 hours.
71. The method of embodiments 1-70, wherein the precipitate is washed with ethyl acetate.
72. The method of embodiments 1-71, wherein the precipitate is washed until achieving a purity of greater than 99%.
73. The method of embodiments 1-72, wherein the precipitate is dried under vacuum at a temperature of from about 20° C. to about 45° C.
74. The method of embodiments 1-72, wherein the precipitate is dried under vacuum at a temperature of from about 20° C. to about 40° C.
75. The method of embodiments 1-72, wherein the precipitate is dried under vacuum at a temperature of from about 20° C. to about 35° C.
76. The method of embodiments 1-72, wherein the precipitate is dried under vacuum at a temperature of from about 20° C. to about 30° C.
77. The method of embodiments 1-72, wherein the precipitate is dried under vacuum at a temperature of from about 25° C. to about 45° C.
78. The method of embodiments 1-72, wherein the precipitate is dried under vacuum at a temperature of from about 25° C. to about 40° C.
79. The method of embodiments 1-72, wherein the precipitate is dried under vacuum at a temperature of from about 25° C. to about 35° C.
80. The method of embodiments 1-72, wherein the precipitate is dried under vacuum at a temperature of from about 25° C. to about 30° C.
81. The method of embodiments 1-72, wherein the precipitate is dried under vacuum at a temperature of from about 30° C. to about 45° C.
82. The method of embodiments 1-72, wherein the precipitate is dried under vacuum at a temperature of from about 30° C. to about 40° C.
83. The method of embodiments 1-72, wherein the precipitate is dried under vacuum at a temperature of from about 30° C. to about 35° C.
84. The method of embodiments 1-72, wherein the precipitate is dried under vacuum at a temperature of from about 30° C. to about 33° C.
85. The method of embodiments 1-84, wherein the reaction mixture is essentially free of water. 86. The method of embodiments 1-84, wherein the reaction mixture contains less than 15% of water.
87. The method of embodiments 1-84, wherein the reaction mixture contains less than 10% of water.
88. The method of embodiments 1-84, wherein the reaction mixture contains less than 5% of water.
89. The method of embodiments 1-84, wherein the reaction mixture contains less than 3% of water.
90. The method of embodiments 1-84, wherein the reaction mixture contains less than 2% of water.
91. The method of embodiments 1-84, wherein the reaction mixture contains less than 1% of water.
92. The method of embodiments 1-84, wherein the reaction mixture contains less than 0.5% of water.
93. The method of embodiments 1-84, wherein the reaction mixture contains less than 0.1% of water.
94. The method of embodiments 1-93, wherein the molar ratio of R′N(CH₃)₂ and the halide according to Formula 2 is about 1:1.
95. The method of embodiments 1-94, wherein the concentration of ethyl acetate is about 190 g/ml to about 200 g/ml.
96. A compound according to Formula 1 made by any one of the methods of embodiments 1-95. 97. A composition consisting essentially of the compound of embodiment 96.
98. The composition of embodiment 97, wherein at least 85% of the composition is the compound of embodiment 96.
99. The composition of embodiment 97, wherein at least 90% of the composition is the compound of embodiment 96.
100. The composition of embodiment 97, wherein at least 95% of the composition is the compound of embodiment 96.
101. The composition of embodiment 97, wherein at least 98% of the composition is the compound of embodiment 96.
102. The composition of embodiment 97, wherein at least 99% of the composition is the compound of embodiment 96.

### NON-LIMITING EXAMPLES

The following examples are given to enable those skilled in the art to more clearly understand and to practice the present disclosure. They should not be considered as limiting the scope of the disclosure, but merely as being illustrative and representative thereof. The following examples are provided to illustrate selected embodiments. They should not be considered as limiting the scope of the disclosure, but merely as being illustrative and representative thereof. Thus, the examples provided below, while illustrated with a particular medical device or active agent, are applicable to the range of medical devices and active agents described herein.

### Example 1

Alkyl dimethyl benzyl ammonium chloride was synthesized as follows: In a 500-ml three-neck RB flask, 50.24 g, 325 mmol ethylbenzyl chloride was mixed with 69.33 g, 325 mmol N,N-dimethyldodecylamine, 200 ml of ethyl acetate, and 200 ppm 2,6-di-tert-butyl-4-methylphenol (BHT) as an inhibitor. The reaction was carried out for 24 hours at 50-55° C. A colorless precipitate was formed. The product was washed with ethyl acetate and dried under vacuum at 30-35° C. water bath. 108.78 g of a white sticky compound was obtained (91% yield). An non-limiting example of the reaction scheme is provided below.

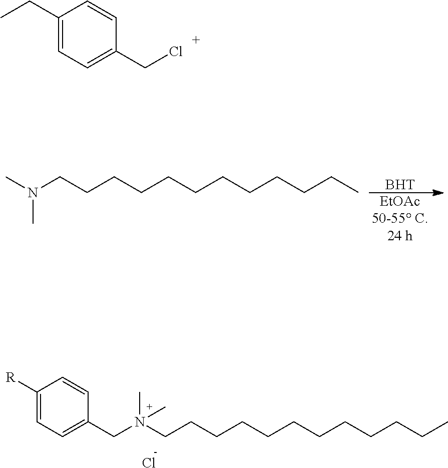

Using a 400 MHZ, CDC13, TMS, the structure of the compound was confirmed: δ 0.88 (t, J=8 Hz, 3H, —CH3), 1.32-1.20 (m, 21H, CH3-CH2-benz, —(CH2)9-), 1.80-1.77 (m, 2H, N+-CH2-CH2-), 2.67 (q, J=8 Hz, 2H, benz-CH2-CH3), 3.30 (s, 6H, —N+-(CH3)2), 3.52-3.48 (m, 2H, —CH2-N+-), 4.99 (s, 2H, benz-CH—N+), 7.24 (d, J=8 Hz, 2H, benzene), 7.55 (d, J=8 Hz, 2H, benzene).

The compound was analyzed using standard reverse phase liquid chromatography/mass spectrometry. Based on peak area at 220 nm, purity was 99.0% with one minor impurity. Mass spectra of the major peak was consistent with the proposed structure. LC/UV/MS was accomplished using an Agilent G6120B MSD (Mass Selective Detector) coupled with an Agilent 1200 HPLC system. Instrument computer operation incorporates Agilent OpenLab CDS software. The MSD is a single stage quadrupole mass spectrometer capable of unit mass resolution with a mass accuracy to ±0.13 Da. The source for the MSD is Electrospray Ionization (ESI). The working mass range for the instrument is 100 to 1500 m/z. Mass calibration of the MSD is performed using an Autotune function which automatically adjusts mass calibration, peak resolution, and intensity.

### Analysis Conditions:

| HPLC/MS | Agilent LC/MSD 6120B | |
|---|---|---|
| Source | Electrospray (ESI) | |
| Ionization Mode | Positive Ion | |
| Method | Gradient Conditions | |
| Mobile Phase A | Water with 0.1% Formic Acid | |
| Mobile Phase B | Acetonitrile/Water with 0.1% Formic Acid | |
| Gradient | 90/10 A/B (0 min) → 10/90 A/B (10 min) → 10/90 A/B (20 min) → | |
| | 90/10 A/B (21 min) → 90/10 A/B (260 min) | |
| Column | Thermo Hypersil Gold 250 × 4.6 mm 3 um SN#1052266 | |
| Temp | 30° | C. |
| Flow Rate | 1 | mL/min |
| Injection Volume | 10 | uL |
| Scan | 75 to 1000 | m/z |
| Step Size | 0.1 | m/z |
| Fragmentor | 80 | V |
| Gain | 0.5 | |
| Threshold | 1 | |
| Drying Gas | 325° | C. |
| Capillary | 3000 | V |

| | |
|---|---|
| Nebulizer Gas | 8 LPM |

### Sample Preparation:

The sample was dissolved to 2.0 mg/mL with 50/50 ACN/H2O.

### Results

One major peak at 9.6 min was noted in the analytical run with nine minor impurities. The area percent for the main compound resulted in a purity of 99.0%. The charged quaternary ammonium ion was noted at 332.3 m/z. This agrees with the isotopic exact mass of 332.3 for the proposed formula of C23H42N.

### Example 2

In a 500-ml three-neck RB flask, 50.24 g, 325 mmol ethylbenzyl chloride was mixed with 69.33 g, 325 mmol N,N-dimethyldodecylamine, 50 ml of ethyl acetate, and 300 ppm 2,6-di-tert-butyl-4-methylphenol (BHT) as an inhibitor. The reaction was carried out for 24 hours at 45-55° C. A colorless precipitate was formed. The product was washed with ethyl acetate and dried under vacuum at 30-35° C. water bath. A white sticky compound was obtained (58% yield).

### Example 3

In a 500-ml three-neck RB flask, 50.24 g, 325 mmol ethylbenzyl chloride was mixed with 69.33 g, 325 mmol N,N-dimethyldodecylamine, 40 ml of ethyl acetate, and 300 ppm 2,6-di-tert-butyl-4-methylphenol (BHT) as an inhibitor. The reaction was carried out for 17 hours at 40-50° C. A colorless precipitate was formed. The product was washed with ethyl acetate and dried under vacuum at 30-35° C. water bath. A white sticky compound was obtained (80% yield).

### Example 4

In a 500-ml three-neck RB flask, 50.24 g, 325 mmol ethylbenzyl chloride was mixed with 69.33 g, 325 mmol N,N-dimethyldodecylamine, 30 ml of anhydrous ethanol, and 300 ppm 2,6-di-tert-butyl-4-methylphenol (BHT) as an inhibitor. The reaction was carried out for 17 hours at 55-65° C. A colorless precipitate was formed. The product was washed with ethyl acetate and dried under vacuum at 30-35° C. water bath. A white sticky compound was obtained (45% yield).

### Example 5

In a 500-ml three-neck RB flask, 50.24 g, 325 mmol ethylbenzyl chloride was mixed with 69.33 g, 325 mmol N,N-dimethyldodecylamine, 200 ml of ethyl acetate, and 200 ppm 2,6-di-tert-butyl-4-methylphenol (BHT) as an inhibitor. The reaction was carried out for 24 hours at 50-55° C. A colorless precipitate was formed. The product was washed with ethyl acetate and dried under vacuum at 30-35° C. water bath. A white sticky compound was obtained.

### Example 6

In a 500-ml three-neck RB flask, equimolar parts benzyl chloride was mixed with N,N-dimethyldodecylamine, 200 ml of ethyl acetate, and 200 ppm 2,6-di-tert-butyl-4-methylphenol (BHT) as an inhibitor. The reaction was carried out for 24 hours at 50-55° C. A colorless precipitate was formed. The product was washed with ethyl acetate and dried under vacuum at 30-35° C. water bath. A white sticky compound was obtained.

### Example 7

In a 500-ml three-neck RB flask, equimolar parts hydrogen chloride was mixed with N,N-dimethyldodecylamine, 200 ml of ethyl acetate, and 200 ppm 2,6-di-tert-butyl-4-methylphenol (BHT) as an inhibitor. The reaction was carried out for 24 hours at 50-55° C. A colorless precipitate was formed. The product was washed with ethyl acetate and dried under vacuum at 30-35° C. water bath. A white sticky compound was obtained.

### Example 8

In a 500-ml three-neck RB flask, equimolar parts ethylbenzyl chloride was mixed with N,N-dimethyldecylamine, 200 ml of ethyl acetate, and 200 ppm 2,6-di-tert-butyl-4-methylphenol (BHT) as an inhibitor. The reaction was carried out for 24 hours at 50-55° C. A colorless precipitate was formed. The product was washed with ethyl acetate and dried under vacuum at 30-35° C. water bath. A white sticky compound was obtained.

### Example 9

In a 500-ml three-neck RB flask, equimolar parts benzyl chloride was mixed with N,N-dimethyloctylamine, 200 ml of ethyl acetate, and 200 ppm 2,6-di-tert-butyl-4-methylphenol (BHT) as an inhibitor. The reaction was carried out for 24 hours at 50-55° C. A colorless precipitate was formed. The product was washed with ethyl acetate and dried under vacuum at 30-35° C. water bath. A white sticky compound was obtained.

### Example 10

In a 500-ml three-neck RB flask, equimolar parts benzyl chloride was mixed with N,N-dimethylhexylamine, 200 ml of ethyl acetate, and 200 ppm 2,6-di-tert-butyl-4-methylphenol (BHT) as an inhibitor. The reaction was carried out for 24 hours at 50-55° C. A colorless precipitate was formed. The product was washed with ethyl acetate and dried under vacuum at 30-35° C. water bath. A white sticky compound was obtained.

### Example 11

In a 500-ml three-neck RB flask, equimolar parts benzyl chloride was mixed with N,N-dimethylbutylamine, 200 ml of ethyl acetate, and 200 ppm 2,6-di-tert-butyl-4-methylphenol (BHT) as an inhibitor. The reaction was carried out for 24 hours at 50-55° C. A colorless precipitate was formed. The product was washed with ethyl acetate and dried under vacuum at 30-35° C. water bath. A white sticky compound was obtained.

### Example 12

In a 500-ml three-neck RB flask, equimolar parts benzyl chloride was mixed with N,N-dimethyleicosylamine, 200 ml of ethyl acetate, and 200 ppm 2,6-di-tert-butyl-4-methylphenol (BHT) as an inhibitor. The reaction was carried out for 24 hours at 50-55° C. A colorless precipitate was formed. The product was washed with ethyl acetate and dried under vacuum at 30-35° C. water bath. A white sticky compound was obtained.

### Example 13

In a 500-ml three-neck RB flask, equimolar parts benzyl bromide was mixed with N,N-dimethyldodecylamine, 200 ml of ethyl acetate, and 200 ppm 2,6-di-tert-butyl-4-methylphenol (BHT) as an inhibitor. The reaction was carried out for 24 hours at 50-55° C. A colorless precipitate was formed. The product was washed with ethyl acetate and dried under vacuum at 30-35° C. water bath. A white sticky compound was obtained.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method making a compound according to Formula 1 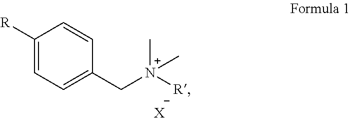
wherein R is hydrogen or a C1-C4 alkyl, wherein R′ is a C4-C22 alkyl, wherein X is a halogen, the method comprising:
(b) dissolving an alkyldimethyl amine R′N(CH₃)₂ and a halide according to Formula 2 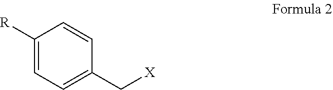
in a solvent to form a reaction mixture, wherein the solvent is anhydrous ethanol, ethyl acetate, or a mixture thereof;
(b) heating the reaction mixture at a reaction temperature less than the reflux temperature of the solvent; and
(c) allowing the reaction mixture to cool, thereby obtaining a precipitate of alkyl dimethyl 4-ethylbenzyl ammonium from the reaction mixture.

2. The method of claim 1, wherein R is ethyl.

3. The method of claim 1, wherein R′ is a C6-C18 alkyl.

4. The method of claim 1, wherein R′ is a C8-C16 alkyl.

5. The method of claim 1, wherein R′ is a C12-C16 alkyl.

6. The method of claim 1, wherein R′ is a C12 alkyl.

7. The method of claim 1, wherein R′ is dodecyl.

8. The method of claim 1, wherein the halide is chloride.

9. The method of claim 1, wherein the reaction mixture in step (b) is heated at a temperature of from about 30° C. to about 75° C.

10. The method of claim 1, wherein the reaction mixture in step (b) is heated at a temperature of from about 35° C. to about 70° C.

11. The method of claim 1, wherein the reaction mixture in step (b) is heated at a temperature of from about 40° C. to about 65° C.

12. The method of claim 1, wherein the reaction mixture in step (b) is heated at a temperature of from about 45° C. to about 60° C.

13. The method of claim 1, wherein the solvent is anhydrous ethanol.

14. The method of claim 1, wherein the solvent is ethyl acetate.

15. The method of claim 1, wherein the heating continues until less than 5% R′N(CH3)2 is present in the reaction mixture.

16. The method of claim 1, wherein the heating continues until less than 1% R′N(CH3)2 is present in the reaction mixture.

17. The method of claim 1, wherein the heating continues until less than 0.1% R′N(CH3)2 is present in the reaction mixture.

18. The method of claim 1, wherein the reaction is carried out for at least 1 hour to about 48 hours.

19. The method of claim 1, wherein the reaction is carried out for at least 1 hour to about 6 hours.

20. The method of claim 1, wherein the precipitate is washed with ethyl acetate.

21. The method of claim 1, wherein the precipitate is washed until achieving a purity of greater than 99%.

22. The method of claim 1, wherein the precipitate is dried under vacuum at a temperature of from about 20° C. to about 45° C.

23. The method of claim 1, wherein the precipitate is dried under vacuum at a temperature of from about 25° C. to about 40° C.

24. The method of claim 1, wherein the precipitate is dried under vacuum at a temperature of from about 30° C. to about 35° C.

25. The method of claim 1, wherein the reaction mixture contains less than 5% of water.

26. The method of claim 1, wherein the reaction mixture contains less than 1% of water.

27. The method of claim 1, wherein the reaction mixture contains less than 0.1% of water.

28. The method of claim 1, wherein the concentration of ethyl acetate is about 190 g/ml to about 200 g/ml.

29. A composition comprising a compound according to Formula 1 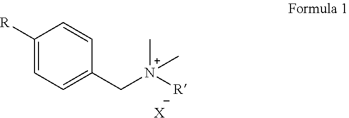
wherein R is hydrogen or a C1-C4 alkyl, wherein R′ is a C4-C22 alkyl, wherein X is a halogen, wherein the composition is made by the method of claim 1.

30. The composition of claim 29, where the composition comprises at least 95% of the compound by weight.
